(19) United States
(12) Patent Application Publication
(10) Pub. No.: US 2016/0312209 A1
(43) Pub. Date: Oct. 27, 2016

(54) Method for Immobilizing and Drying Enzymes

(71) Applicant: BASF SE, Ludwigshafen (DE)

(72) Inventors: Steffen Maurer, Hirschberg (DE); Robert Bayer, Sinsheim (DE); Michael Budde, Ilvesheim (DE); Michael Kerber, Weinheim (DE); Farzad Farivar-Memar, Limburgerhof (DE); Jürgen Däuwel, Heidelberg (DE); Thomas Berg, Ludwigshafen (DE); Sascha Rollie, Mannheim (DE)

(21) Appl. No.: 15/103,899

(22) PCT Filed: Dec. 8, 2014
(86) PCT No.: PCT/EP2014/076844

§ 371 Date: Jun. 13, 2016

(30) Foreign Application Priority Data
Dec. 16, 2013 (EP) 13197469.3

(51) Int. Cl. C12N 11/08; C12N 9/20

(52) U.S. Cl. CPC C12N 11/08; C12N 9/20; C12Y 301/01003

(57) ABSTRACT

Method for immobilizing proteins on a support, wherein the protein is incubated with the support in an aqueous phase in a discontinuous contact vacuum mixer dryer and the immobilized protein is then immediately dried, optionally following an optional washing step, in the same contact vacuum mixer dryer.

### DESCRIPTION OF THE INVENTION

The invention relates to an improved method for immobilizing and drying proteins, particularly enzymes, in particular lipases.

### PRIOR ART

EP382767 describes a method for immobilizing lipases. In this case, an aqueous solution of a given lipase is mixed by rotation with a resin (for example Lewatit®) at fixed pH at room temperature. The resin with immobilized lipase was then collected by filtration, followed by washing with water and drying under reduced pressure.

The immobilization of enzymes is generally accompanied by loss of enzyme as a result of non-binding on the support or by desorption (“bleeding”) of enzyme already bound. Furthermore, the immobilized enzyme frequently experiences a loss of enzyme activity during the method steps of immobilization, which leads to significant yield losses and associated increased costs, especially on an industrial scale.

### DESCRIPTION OF THE INVENTION

The object of the invention, therefore, was to find a method which allows an efficient immobilization of the protein on the support to be achieved, which signifies that as much as possible of the entire amount of enzyme present on the support is permanently bound and remains and, in the case of an enzymatically active protein, as far as possible the same high enzymatic activity is obtained after immobilization as before immobilization.

A method for immobilizing proteins on a support has been found, wherein the protein is incubated with the support in an aqueous phase in a discontinuous contact vacuum mixer dryer and the immobilized protein is then immediately dried, optionally following an optional washing step, in the same contact vacuum mixer dryer.

It has now been found that the method defined at the outset leads to particularly advantageous results since the overall steps of the immobilization, such as efficient incubation of protein with support, optionally washing the immobilized protein and drying the immobilized protein, are carried out in a single apparatus to give a stable product which can be readily stored and transported. This method is particularly well suited to an industrial scale, if immobilized protein is to be produced on a scale of hundreds of kilograms up to a tonne scale.

Using the method according to the invention, numerous proteins may be immobilized. It is particularly suitable for immobilizing enzymes such as oxidoreductases, hydrolases, isomerases and transferases.

It is particularly well suited for immobilizing hydrolases, in particular lipases.

Within the lipases, those lipases from Candida antarctica can be particularly effectively immobilized, particularly Candida antarctica lipase B (CALB) or such enzymes which are structurally derived therefrom. Such lipases structurally derived from CALB are lipases having at least one, preferably two or more amino acid alterations such as insertions, deletions or substitutions, compared to the CALB polypeptide sequence.

Examples of such lipases structurally derived from CALB are described in WO 2009/080676 (“CALB muteins”), wherein the disclosure content with respect to the CALB muteins in WO2009/080676 is explicitly incorporated by reference.

The enzymes to be immobilized may be isolated from the original organism by known methods or also may be produced by recombinant DNA techniques in suitable host organisms such as Bacillus, E.coli, Pichia, Chrysosporium, Aspergillus and Saccharomyces.

Suitable supports are various organic or inorganic materials such as silica gel, activated carbon or polymeric supports. Suitable polymeric supports are macroporous crosslinked polymers having a particle size of 100 to 1000 ∥m and an average pore radius of 10-20 nm. Particularly suitable are macroporous crosslinked acrylate polymers such as poly(meth)acrylates crosslinked with divinylbenzene, which may comprise, for example, acrylic acid, acrylic ester, methacrylic acid and methacrylic ester. Such polymers are sold, for example, by Lanxess under the name Lewatit® VP OC 1600 or by DOW under the name Amberlite® XAD-7.

Suitable discontinuos contact vacuum mixer dryers are known to those skilled in the art from the literature (e.g. Friedrich Kneule, “Das Trocknen” [Drying], Sauerländer A G, Aarau, 1975, ISBN 3-7941-0429-3). Particularly advantageous is the use of a vacuum tumble dryer or double-cone dryer. Well suited are tumble dryers having an internal volume of greater than 10 liters, preferably greater than 100 liters, preferably greater than one cubic meter.

The protein to be immobilized is incubated with the support in an aqueous phase adjusted to a particular pH, generally by buffer.

The pH depends on the nature of the enzyme to be immobilized, mainly on the isoelectric point of the enzyme. A pH range of 3 up to 11, particularly from 4 to 8, has proven to be advantageous.

A pH range of 4.8-5.2 is recommended for CALB lipases.

Suitable buffers for the aqueous phase are, for example, phosphate and acetate buffer.

The incubation is carried out generally at a temperature from 0° C. to 40° C., in particular from 4° C. to 30° C. If the enzyme to be immobilized is particularly tolerant to temperature, even temperatures above 50° C. may be suitable.

After the incubation, the enzyme-containing starting solution is separated from immobilized enzyme. This is most simply carried out by filtration through a sieve plate which is installed in the tumble dryer. The immobilized enzyme can subsequently be purified, if desired, in a washing step. Such washing steps are also carried out by mixing the immobilized enzyme with the washing solution, generally water, and subsequently separating from the immobilized enzyme via an integrated filtration unit.

The immobilized enzyme is subsequently dried also without further transferring into the discontinuous contact vacuum mixer dryer in which a reduced pressure is set in the dryer of less than 1013 mbar, preferably less than 100 mbar.

The jacket temperature is adjusted to less than 100° C., preferably less than 65° C., wherein it must be ensured that the product temperature does not exceed 50° C., preferably 40° C.

It is generally recommended not to set the temperature of the immobilized enzyme to be dried above 50° C. in order to avoid thermal inactivation of the enzyme. In cases of particularly temperature-insensitive enzymes, however, they can even be dried at above 50° C.

Under the conditions specified above, the drying time is typically from 10 to 30 hours, preferably from 15 to 20 hours.

The aim generally is to provide an immobilized product having a residual moisture content of less than 5%, preferably less than 2% and particularly preferably from 0.5 to 1.5% water.

If the product is excessively dry, this results in static charge which partially impedes the handling.

A product produced using the method according to the invention is easy to store at room temperature without notable losses of activity occurring. Furthermore, it is easy to handle, i.e. it can be readily filled and transferred.

The novel method is described in more detail in the example below.

### EXAMPLE

Immobilization of CALB on Lewatit® on a 500 kg scale

The T0055 tumble dryer used for this method has the following technical data:
Volume: 6.3 m³
Heating surface: 17 m²

### 1. Raw Materials Used

The following components are required for the experimental procedure:

Lewatit® VP OC 1600 moist (Lanxess) [544 kg]
Lipase ultrafiltrate CALB-2012-01 [21 kg of protein]
Demin. water (for 3 washing steps [3×2000 L]
N₂ stripping gas [5-20 Nm³/h]

According to protocol, a total of 544 kg of moist Lewatit should be filled. This 544 kg of moist Lewatit correspond to about 233 kg of dry Lewatit.

A specific activity of 460 TBU/mg of protein and a purity of 61% for the lipase is apparent.

The whole amount of 21 kg of enzyme is used for the immobilization.

### 2. Experimental Procedure

Feeding of the Tumble Dryer with Lewatit® and First Washing Step
at 08:15 the weighing of the Lewatit drums and then transfer via a funnel into the tumble dryer was initiated
a total of 544.1 kg of moist Lewatit VP OC 1600 were placed in the dryer a mixed sample was collected from all drums
2000 L of demineralized water (via water meter) was then fed via a tube also through the funnel into the dryer at a flow rate of 80-90 l/min →in this manner the funnel was freed of Lewatit residues
the filling process was completed at 08:55
by observation in the dryer, it could be seen that the water had a slightly milky discoloration and that some proteinaceous foam had formed on the surface
the sieving unit (160 μm) was now mounted on the manhole cover (09:20)
the dryer was then evacuated to 78.6 mbar for leak testing
the vacuum was then released to return to standard pressure (1020 mbar) (09:30)
the mixture was then mixed for 2 hours at standard pressure, room temperature and at 0.33 rpm (for this purpose the drive motor on the tumble dryer was set to 5.5 Hz, which corresponds to 0.33 rpm→manually remeasured: 1 rev /2.43min =0.41 rpm) →the heating bath was set at full cooling
after the course of 2 hours (11:26) the T0055 was positioned with the lid downwards and the wash water was filtered off via the incorporated 160 μm sieve into IBCs →it was filtered at a positive pressure of 100-200 mbar →the positive pressure was generated by the nitrogen feed
a sample was taken in each case after 20 L, 1000 L and 1700 L →purely visually, the first sample had a slight cloudiness, which did not settle at the bottom however →the second and third samples were clear; small individual suspended particles were visible
   against the light
a total of 1700 L of water were discharged (11:30-12:25)
the IBCs were allowed to stand for 2 hours
it was subsequently checked whether the suspended particles had settled out at the bottom;
   but this was not the case
the IBCs filled with wash water were weighed
   IBC 1: 1003 kg net
   IBC 2: 717 kg net
the water was then discharged into bbA

### Second Washing Step

the manhole was again positioned upwards
subsequently, 2000 L of water (2000 kg), which were already in two 1000 L IBCs, were aspirated at 800 700 mbar abs. (12:40-13:22)
the filling process took about 20 minutes per container
the mixture was then mixed for 2 hours in the tumble dryer at 0.33 rpm (5.5 Hz), at room temperature and standard pressure
after the course of 2 hours the manhole was positioned downwards and the wash water was filtered off via the incorporated 160 μm sieve into IBCs →this was filtered again at a positive pressure of 100-200 mbar
a sample was taken in each case after 50 L, 1000 L and 2000 L
in total about 2050 L of water were discharged
the filtration took about 30 minutes per container (i.e. per 1000 L)
the IBCs filled with wash water were then weighed
   IBC 1: 1002 kg net
   IBC 2: 1043 kg net
the wash water was then discharged into bbA
a sample of the washed Lewatit was then extracted →it became apparent here that after stopping the rotation of the dryer, the filter cake stood slightly at an angle on the inner wall
the tumble dryer was rotated overnight once every hour to avoid the formation of lumps

### Lipase Addition and Immobilization

the cooled lipase drums supplied were firstly weighed once again →1749.6 kg of lipase net
a 2 L sample was then withdrawn from each drum (09:30-09:50)
the entire 9 drums (200 L each) of lipase CALB-2012-01 were filled into the tumble dryer
to this end, the content of each of 3 drums was sucked into the dryer
for this purpose a reduced pressure of 300 mbar was applied
firstly, containers 7,8,9 were charged (10:19-10:32)
only a very low amount of enzyme solution remained in the drum →after addition of the content of each of 3 drums, the dryer was rotated every 10 minutes at 0.33 rpm
subsequently, the content of containers 3,2,1 was sucked in (11:06-11:23) and rotated at 0.33 rpm every 10 minutes
finally, the content of containers 6,5,4 was sucked in (11:52-12:07)
from 12:20 to 13:00, the drum was rotated at 200 mbar abs. and 0.33 rpm
at 13:00, the tumble dryer was degassed down to 900 mbar abs.
subsequently, until 13:15, the content was further mixed at a reduced pressure of 100-200 mbar →the internal temperature here was 20° C.; for this reason, the cooling, which was set to 24° C., was turned off
then a slight positive pressure of 100-200 mbar was applied in the dryer
under these conditions, the dryer was in motion at 0.33 rpm for 15 hours
the pressure and temperature were recorded hourly
after the first 3 hours (15:15), the first sample (250 ml) was taken from the supernatant of the immobilizate and stored refrigerated at 8-14° C.
in total, samples were taken after 3, 5, 7, 9, 12 and 15 hours immobilization
after 15 hours, the dryer was switched off and rotated only once every hour (1 rotation) (until the next day, 07:00)

### Filtration of the Enzyme-Lewatit Solution After Immobilization

at 07:00 the tumble dryer was rotated again and positioned with the manhole cover downwards
a positive pressure of around 100 mbar was applied in the dryer
the mother liquor was then allowed to drain through the 160 μm sieve into IBCs (07:10-08:20)
after about 300 L, a first sample was withdrawn into a glass flask →it was noticed here that the sample was cloudier and no longer quite as dark-green as the previous day and also foamed →the sample was thus optically brighter and more cloudy than that of the previous day, and now also smelt differently (of butyric acid)
after about 900 L, the second sample was likewise withdrawn into a glass flask
since the mother liquor foamed markedly on filling into the IBC, the first IBC could only be filled with 900 L of mother liquor
then the second IBC was likewise filled with about 700 L of mother liquor, which was firstly purged with nitrogen
for this reason the taps were closed and left for 10 minutes to give the mother liquor time to settle out at the bottom
the remainder of the mother liquor was then added to the IBC
here, a third sample was withdrawn into a glass flask
at the end, the second IBC was filled with a total of 750 L of mother liquor
finally, both IBCs were weighed →IBC 1+2=1683 kg net

### Washing the Immobilized Lipase

after draining the mother liquor, the tumble dryer was firstly rotated once again and positioned with the slide upwards
a sample of the moist, unwashed immobilizate was then taken via the slide →the immobilizate had again dropped off the sides of the dryer →the immobilizate had a pale green tint
then the dryer was again rotated such that the manhole cover was upwards
a reduced pressure of about 300 mbar was applied in the dryer
2000 L of demineralized water (bottled in 2 IBC) were then sucked into the tumble dryer (08:55-09:30)
2 hours of mixing then followed at 0.33 rpm, room temperature and standard pressure
after 2 hours, the manhole cover was positioned downwards
a positive pressure of 400-580 mbar was applied in the dryer and the wash water was discharged into IBCs (11:45-13:15)
the first sample was taken after 50 L, the second after 1000 L and the third after 1825 L →all samples were slightly cloudy greenish; but no sediment settled out →no major difference between the samples could be seen
after about 1750 L wash water had been discharged, the mixture was firstly purged with N₂
after about 1850 L of wash water had been discharged, a third IBC was placed outside and the line attached to the outside since very large amounts of nitrogen had been purged
a positive pressure of 900 mbar was now applied to the dryer in order to effectively filter out the total amount of water
at this pressure, the mixture was blown dry for 15 minutes (at the end only nitrogen came out of the line and occasional drops of wash water)
subsequently, the IBCs were weighed →IBC 1+2=1921 kg net
a 1 L sample of solid was then taken via the manhole →this sample also was very light greenish (but not so pronounced as the solid sample taken previously)

### Vacuum Drying

the tumble dryer was initially evacuated under rotation at 0.33 rpm
the rotation was then increased stepwise to 2.4 rpm (1/1.5/2.0/2.4 rpm) →the dryer was very quiet considered from the outside which in turn suggests a uniform sliding (slipping) of the immobilizate from the dryer wall (at this time point therefore no relatively large lumps had formed)
at 14:20 the heating was set to 50° C.
at 17:30 it was again closed and instead the stripping nitrogen was maintained at 25 m³/h
at 02:15, the cooling was adjusted in order to be able to take the first sample after a cooling time of 2 h, the first sample of immobilizate was taken at 04:15 →the internal temperature had decreased from 39.3° C. to 23° C.
the sample was given to the company's own laboratory for residual moisture determination →residual moisture determination at 105° C./60 minutes: 35.76%
at 08:25 the dryer was positioned with the slide downwards and allowed to stand for 5 minutes to measure the temperature →the internal temperature had a temperature difference of 5° C., depending on whether the tumble dryer is rotating or whether the measuring sensor is dipped into the solid
at 13:45, the cooling was adjusted in order to be able to take a second sample
after a cooling time of 30 minutes, the sample was then taken →it was noticed here that there were also smaller, dark (brown) particles between the white beads of the immobilizate
the residual moisture was then determined in the company's own laboratory →residual moisture determination at 105° C./60 minutes: 9.72%
sample then stored in the refrigerator
at 17:28 the internal temperature increased to 46° C. >bath set to 50° C.
at 17:47 the internal temperature had fallen back to 45.5° C. >bath set to 53° C.
from 18:00 the cooling was started
from 19:00-19:20, the sample was taken and brought to the company's own laboratory for residual moisture determination →residual moisture determination at 105° C./60 minutes: 1.4%
since the residual moisture was below the target value of 3%, the drying was terminated
at 20:30 the vacuum was released →here the temperature increased from 25° C. to 31.7° C.
at 20:50 the drive was switched off at an internal temperature of 25.8° C.
from this time point, the dryer was rotated once every 2 hours

### Bottling

the dryer was positioned with the slide downwards
the sieving unit was then again detached →slight product residue on the sieve
manhole cover left partly open for bottling procedure
Nibbler/adapter mounted
the dryer then inertized with about 2.4 m³/h N₂
Big Bag No. 67291120 hung on the filling device and purged with N₂ and grounded
exhaust on filling device half open
by means of manual tapping, product is intially allowed to carefully drain out; at a later stage rotated to about a half position →the immobilizate is sprinkled without difficulty into the suspended Big Bag, without remaining on the film or in the dryer itself →the Big Bag was also only once very briefly inflated as some more product came out for a brief moment
the residues, which were still in the tumble dryer, were then tapped out →no major residues remained overall in the tumble dryer (only 3 locations of about DIN A3 size, to which perhaps a 2 mm thick layer had formed; otherwise the dryer inner wall was only coated with a very fine dust layer)
another 1 L sample was taken in a plastic flask from the big bag
finally, the Big Bag was again weighed and subsequently stored dry →Tare: 25 kg →Gross: 271 kg
the filling procedure itself took only about 5 minutes (together with the preparation it was about 20 minutes)

### 3. Analytical Results

A theoretical absolute activity of 9 460 000 000 TBU was calculated at the start

### Measured Activity/Total Protein in the Course of the Immobilization:

| | | | Residu- | | |
|---|---|---|---|---|---|
| | | | al activi- | | |
| | | | ty in the | Total | Total |
| | | | SN [%] | protein | protein |
| | Activity | Total activity | from | [g/L] | [%] |
| | volume | [TBU] | starting | CooPlus/ | CooPlus/ |
| Sample | [TBU/mL] | abs. calc. | value | BGG | BGG |
| Mixed sample concentrate | 5390 | 9,270,800,000 | | 8.63 | |
| from drums CALB-2012-01 | | | | | |
| mixed corresp. to the | | | | | |
| resp. masses | | | | | |
| (kg net) of the drums | | | | | |
| Supernatant after 3 h im- | 2215 | 4,474,300,000 | 48 | | |
| mobilization, native | | | | | |
| Supernatant after 3 h im- | 2225 | 4,494,500,000 | 48 | 3.47 | 40.16 |
| mobilization, filtered | | | | | |
| (PVDF 0.2 μm) | | | | | |
| Supernatant after 5 h im- | 1730 | 2,975,600,000 | 32 | | |
| mobilization, native | | | | | |
| Supernatant after 5 h im- | 1760 | 3,027,200,000 | 33 | 3.13 | 36.23 |
| mobilization, filtered | | | | | |
| (PVDF 0.2 μm) | | | | | |
| Supernatant after 7 h im- | 1220 | 2,098,400,000 | 23 | | |
| mobilization, native | | | | | |
| Supernatant after 7 h im- | 1090 | 1,874,800,000 | 20 | 1.79 | 20.68 |
| mobilization, filtered | | | | | |
| (PVDF 0.2 μm) | | | | | |
| Supernatant after 9 h im- | 820 | 1,410,400,000 | 15 | | |
| mobilization, native | | | | | |
| Supernatant after 9 h im- | 744 | 1,279,680,000 | 14 | 1.45 | 16.85 |
| mobilization, filtered | | | | | |
| (PVDF 0.2 μm) | | | | | |
| Supernatant after 12 h | 502 | 863,440,000 | 9.3 | | |
| immobilization, native | | | | | |
| Supernatant after 12 h | 460 | 791,200,000 | 8.5 | 0.85 | 9.88 |
| immobilization, filtered | | | | | |
| (PVDF 0.2 μm) | | | | | |
| Supernatant after 15 h | 443 | 761,960,000 | 8.2 | | |
| immobilization, native | | | | | |
| Supernatant after 15 h | 425 | 731,000,000 | 7.9 | 0.92 | 10.69 |
| immobilization, filtered | | | | | |
| (PVDF 0.2 μm) | | | | | |
| Supernatant at the end of | 204 | 411,070,000 | 4.3 | | |
| the immobilization, native | | | | | |
| Supernatant at the end of | 174 | 351,480,000 | 3.7 | 0.43 | 4.97 |
| the immobilization, fil- | | | | | |
| tered (PVDF 0.2 μm) | | | | | |
| Wash water 3 after im- | 20.4 | | | | |
| mob., sample 1 native | | | | | |
| Wash water 3 after im- | 17.1 | | | 0.06 | |
| mob., sample 1 filtered | | | | | |
| (PVDF 0.2 μm) | | | | | |
| Wash water 3 after im- | 35.8 | | | | |
| mob., sample 2 native | | | | | |
| Wash water 3 after im- | 19.5 | | | 0.06 | |
| mob., sample 2 filtered | | | | | |
| (PVDF 0.2 μm) | | | | | |
| Wash water 3 after im- | 28.0 | | | | |
| mob., sample 3 native | | | | | |
| Wash water 3 after im- | 33.4 | | | 0.07 | |
| mob., sample 3 filtered | | | | | |
| (PVDF 0.2 μm) | | | | | |

An immobilization efficiency of 96.3% was achieved.

### Residual Moisture Determination in the Course of the Vacuum Drying

| | Residual moisture content |
|---|---|
| Drying time [h] | [%] |
| 0 | 53.1-74.2 |
| 12 | 35.76 |
| 18 | 9.72 |
| 22 | 1.40 |

### Residual Moisture Content of the Bottled Product

With the aid of the infrared balance, a triplicate measurement of the average dry content of the sample, taken from the filled Big Bag, was determined.

The measurements gave the following result (infrared balance, end point: 30 sec. scale stability, triplicate measurement: 5.3 g/9.3 g/4.6 g):

The average dry content of the filled product is 99.11%.

This corresponds in turn to a residual moisture content of 0.89%.

1. A method for immobilizing a protein on a support, wherein the protein is incubated with the support in an aqueous phase in a discontinuous contact vacuum mixer dryer and the immobilized protein is then immediately dried, optionally following an optional washing step, in the same contact vacuum mixer dryer.

2. The method according to claim 1, wherein the contact vacuum mixer dryer used is a tumble dryer having an internal volume of more than 100 L.

3. The method according to claim 1, wherein the protein used is an enzyme.

4. The method according to claim 3, wherein the enzyme used is a lipase.

5. The method according to claim 4, wherein the lipase used is Candida antarctica lipase B (CALB) or a lipase structurally derived therefrom.

6. The method according to claim 1, wherein the support used is a material having a hydrophobic surface.

7. The method according to claim 1, wherein the polymeric support used is a crosslinked poly(meth)acrylate-containing resin.

8. The method according to claim 7, wherein the polymeric support used is a macroporous poly(meth)acrylate resin crosslinked with divinylbenzene in spherical bead form, whose particles have at least 60% of the mass and a size of 50 μm to 2000 μm.

9. The method according to claim 1, wherein the incubation time of the protein with the support is 2-30 hours.

10. The method according to claim 1, wherein the protein is incubated with the support at a temperature of from 0° C. to 40° C.

11. The method according to claim 1, wherein the drying temperature of the immobilized protein is 30-60° C.

12. The method according to claim 1, wherein the drying is carried out under reduced pressure.

13. The method according to claim 12, wherein the drying is carried out in a range of 5 to 800 mbar.
